(19) United States
(12) Patent Application Publication
(10) Pub. No.: US 2005/0250088 A1
(43) Pub. Date: Nov. 10, 2005

(54) Cryopreservation media

(76) Inventor: Jeffrey P. Boldt, Carmel, IN (US)

Correspondence Address: LATHROP & GAGE LC, 2345 GRAND AVENUE, SUITE 2800, KANSAS CITY, MO, 64108, US

(21) Appl. No.: 11/090,581

(22) Filed: Mar. 25, 2005

Related U.S. Application Data
(60) Provisional application No. 60/556,172, filed on Mar. 25, 2004.

(51) Int. Cl. A01N 1/02; C12N 5/08

(52) U.S. Cl. 435/2; 435/366

(57) ABSTRACT

A sodium-depleted media that does not employ PBS as the buffer for use in the cryopreservation of oocytes is provided. Also provided are methods of using said media and oocytes cryopreserved therewith.

### RELATED APPLICATION

This application is the nonprovisional application and claims priority to U.S. Patent Application Ser. No. 60/556,172, filed Mar. 25, 2004, the disclosure of which is incorporated by reference herein.

### FIELD OF THE INVENTION

This invention relates to media employed in the cryopreservation of mammalian cells, including oocytes.

### BACKGROUND OF THE INVENTION

The ability to cryopreserve mammalian oocytes with high success rates in an easily reproducible manner has not yet been achieved. Cryopreservation of mouse oocytes, for example, could be valuable for the long term storage of genetically important strains of mice and could additionally serve as a model for mammalian oocyte cryopreservation, including in humans, commercial livestock, and endangered species. Although offspring have been produced from frozen-thawed oocytes in the mouse, human, bovine, and rabbit (Whittingham, 1977; Al-Hasani et al., 1987; Fuku et al., 1992; Chen 1986; Van Uem et al., 1987), results have been variable and not sufficiently successful to make oocyte cryopreservation routine.

Human oocyte survival rate in cryopreservation depends both on the size of the oocyte and the cryoprotectant used, including the composition, concentration and exposure time, and the freezing/thawing rate.

In the cryopreservation process, oocyte size is a very important parameter affecting the survival rate because the large quantity of water in ooplasm causes intracellular ice formation during the freezing process: intracellular ice is one of the main responsible factors for intracellular structure damages.

Oocyte cryopreservation protocols usually includes initially exposing the oocytes to a solution including a permeating cryoprotectant (e.g. 1,2-propanediol (PROH)), which functions to reduce to a minimum intracellular structure damages caused by water crystallization; subsequently exposing for a time of 2-5 min. the oocyte to a so-called loading solution including a mixture of a permeating cryoprotectant and a non permeating cryoprotectant (e.g. sucrose) to increase oocytes dehydration; slowly cooling to −150° C.; storing in liquid nitrogen (−196° C.); thawing, and diluting and removing the cryoprotectants by exposure to so-called thawing solutions and returning to the physiological environment for further manipulations.

In addition to oocyte freezing, cryopreservation and transplantation of ovarian autografts have been somewhat successful in mice and sheep (Gosden et al., 1994; Gunasena et al., 1997). The ovarian tissues in these reports were frozen using simple embryo freezing protocols. Ovarian tissue survived freezing and was able to continue development after transplantation to the reproductive tract or kidney capsule, yielding growing follicles.

Current cryopreservation protocols have evolved from embryo freezing methods that produced offspring in mice, cows, and sheep (Whittingham et al., 1972; Willadsen et al., 1976, 1978). Cryopreservation procedures for mouse and other mammalian embryos are now relatively efficient, but these techniques cannot be used reliably for oocyte freezing. Studies cryopreserving mouse oocytes report very different survival and fertilization rates (Carroll et al., 1993; Carroll et al., 1990; Cohen et al., 1988; George et al., 1994; Glenister et al., 1990; Gook et al., 1993; Whittingham et al., 1977). The variability in the success of mouse oocyte freezing is most likely due to modifications in freezing protocol and/or the use of different cryoprotectants. Although these protocols differ, they rely on the same basic cryobiological principals.

A particularly important area that would benefit greatly from advances in cryopreservation technology is assisted human reproduction. In this field, one or both partners may have a fertility problem. In order to overcome these problems, eggs are harvested from the mother, and sperm from the father. The sperm are then used to fertilize the eggs, and one or more developing embryos are then replaced to the uterus of the mother. Typically, egg maturation is induced pharmaceutically prior to harvesting, and the sperm must be available to fertilize the eggs. Generally, only a very limited number of surgical harvesting procedures may be conducted on an individual, and the number of eggs replaced in the mother is limited in order to avoid multiple live births. Therefore, there has been a need to preserve harvested eggs, sperm, fertilized eggs, and embryos.

The ability to successfully and reproducibly cryopreserve oocytes, which is not yet possible, would allow women to have their eggs frozen until a time when they have found a suitable sperm donor (possibly a future husband) and then thaw their frozen eggs and fertilize them. The resulting embryos can be replaced into the patients' uterus, after it has been prepared to receive the embryo using hormones and known techniques, to allow for implantation, fetal development, and ultimately birth. Ethically, there are issues involved in preserving fertilized eggs or embryos, that are more significant than those associated with freezing unfertilized germ cells. Cryopreservation of oocytes would avoid the ethical concerns surrounding embryo freezing in humans and offer another option to couples with infertility problems. Storing oocytes early in life, such as when the mother is in her 20's and early 30's, when healthy eggs tend to be produced, and therefore when they are more apt to be fertilized and result in viable offspring, would greatly improve the chance of a pregnancy later in life, rather than the relatively poor pregnancy rates produced when participating in an in vitro fertilization (IVF) program at 40 years old or older. Viable cryopreserved oocytes result in healthy embryos and offspring thereby.

Cryopreservation of oocytes, especially from humans, in a reproducible and efficient manner has been generally unsuccessful according to known techniques. It is noted, however, that offspring have been produced from frozen oocytes of several species, including humans. An improved cryopreservation medium would benefit oocyte storage and may also provide a more successful way of freezing embryos, thereby improving the possibilities for pregnancy.

Cryopreservation technology is applicable to other species besides humans. In commercial livestock, improvements in oocyte or embryo cryopreservation could greatly improve genetic management of populations and the number of offspring generated, resulting in a significant time savings and efficiency. In endangered animals, any improvement in embryo freezing or the development of a method to freeze eggs could lead to an increase in the number of offspring produced, enhancement of the genetic quality of the offspring, improvement of the population's genetic health, elimination of both the cost and risk of transporting live animals for reproductive purposes, and possibly even a delay or prevention from extinction of certain species.

Cryopreservation of oocytes from endangered species would provide an invaluable method of salvaging important genetic material. Provided that the eggs could be thawed, fertilized, and produce fertile offspring, and given that sperm is relatively easy to store, species could be stored indefinitely, virtually eliminating the risk of extinction. Cryopreserved oocytes could be easily transported globally, providing a source of genetic information to better aid in managing populations. In this manner, underrepresented genes from founder animals could be reintroduced into the population at any time, even 200 years from now. Because frozen oocytes (and sperm) can be distributed easily and cost effectively, the possibilities of improving genetic diversity and the overall health of a population are intriguing. Oocytes collected in the field can be frozen and infused into the captive population to improve its genetic health. With this technology in place frozen zoos can become a reality.

Biological cryopreservation systems are well known. These systems allow cells to be frozen, for example at −20° C. or below, for extended periods, and resume normal cell activity after thawing. Typically, problems encountered include intracellular ice formation (IIF) and osmotic imbalances that result in cellular disruption. Many prior methods are directed to the prevention of IIF.

Cryopreservation of cells involves dehydration, introduction of a cryoprotectant, and cooling to a low temperature, usually from −30° C. to −80° C., before plunging in LN₂. The first objective is the removal of water from the cell, which when cooled below its melting point forms ice crystals that can damage intracellular organelles as well as the cell membrane (Mazur, 1977). The osmolality of the extracellular solution increases as the outside water freezes, causing the water to passively exit the cell. More ice forms at lower temperatures resulting in continued cellular dehydration. The next objective for freezing cells concerns the combining of any residual water left in the cell with a cryoprotectant, in order to form a glass-like structure when solidified, thereby preventing IIF. Because the melting point of water is reached both during freezing and thawing, IIF can occur at either time. Damage may therefore occur when the cell is exposed to elevated concentrations of electrolytes and/or IIF. IIF can be catalyzed by the presence of extracellular ice surrounding the cells (seeding) or heterogeneously by intracellular structures. In the presence of cryoprotectants, however, IIF resulting from either seeding and/or heterogeneous ice nucleation does not occur (Toner et al., 1991), suggesting that IIF may not be a problem when freezing oocytes in the presence of a cryoprotectant. Therefore, electrolytes including sodium would appear to impart the majority of the damage during cryopreservation. Cellular disruption by sodium ions could alter the cell membrane and/or intracellular organelles. Lovelock (1954) hypothesized that cellular damage is caused by an increase in electrolyte concentration, causing destabilization of membranes. Mazur et al. (1974) further demonstrated that the cell surface is a major site of freezing damage. In his study, Mazur showed that the non-permeating solute sucrose was as effective in protecting erythrocytes from freeze/thaw damage as the permeable cryoprotectant glycerol. The majority of damage to mouse oocytes during freezing may be caused by sodium ions, but we cannot rule out the possibility of IIF. Whether cellular demise is caused by exposure to elevated sodium ion concentrations during the freeze or thaw, or a problem with the transport of sodium ions across cell membranes remains to be elucidated. Sodium ions have a radius of about 0.95 Å, while lithium has a radius of 0.60 Å and potassium has a radius of about 1.33 Å The majority of past cryopreservation studies that have focused on IIF, cryoprotectants, and altering freezing protocols have been unable to significantly improve oocyte freezing. Therefore, the type of cryoprotectant used, or the freezing protocol may already be adequate for oocyte freezing and IIF may not be the major problem presumed by in earlier methods.

Standard embryo cryopreservation techniques are known. In general, embryos are exposed to a cryoprotectant (dimethyl sulfoxide (DMSO), 1,2-propanediol (PrOH), glycerol, ethylene glycol), diluted in a simple sodium-based salt solution for 5-15 min to allow uptake of the cryoprotectant. The embryos are then cooled quickly (−2° C./min) to about 7° C. at which point they are seeded, cooled slowly (−0.3° C. to −0.5° C./min) to about −30° C. or below, and then plunged directly into liquid nitrogen (LN₂). Embryos can also be rapidly frozen or vitrified, but only using very elevated cryopreservative concentrations (2M to 6M) that are toxic to cells when they are exposed for more than a few minutes. Following cryopreservation the embryos are thawed and cultured. Thawing procedures differ, but very little. Two basic concepts are involved in the thawing process, 1) removal of cryoprotectant and 2) rehydration of the embryo at a rate so as not to rupture the cell membrane, usually with the use of sucrose. These freezing and thawing procedures work relatively well for embryos, but do not allow the successful storage of oocytes. The exact reason(s) why embryos can survive and oocytes cannot are unknown, but membrane damage due to IIF, ion loading, and/or osmotic stress are suspect. Researchers have mainly focused on the problem of IIF and osmotic stress by modifying freezing procedures (slow vs. fast), thawing procedures (slow vs. fast), and methods for the removal of the cryoprotectants with little attention to the cryopreservation media formulation, which is presumed to be optimal.

Researchers have tried unsuccessfully to cryopreserve oocytes in a reproducible manner using one or more cryoprotectants. In general, PROH and DMSO are the most common cryoprotectants used today for the cryopreservation of oocytes and embryos. Although pregnancies in the human have resulted from oocytes frozen in DMSO and PrOH, PrOH is the cryoprotectant of choice because of its greater permeability, reduced toxicity, and improved success in storing human embryos (Gook et al., 1995; Imoedemhe and Sigue, 1992; Lassalle et al., 1985; Mandelbaum et al., 1987; Testart et al., 1986). Mouse oocytes have been frozen using PrOH, but with poor overall results (Gook et al., 1993; Todorow et al., 1989). Todorow et al. (1989) reported survival and fertilization rates of 63% and 27%, respectively. When PROH was used in combination with DMSO, survival and fertilization rates increased to 87% and 42%, respectively. Numerous studies have reported survival and fertilization rates of up to 79% and 50%, respectively, for mouse oocytes cryopreserved using DMSO (Glenister et al., 1987; George and Johnson, 1993; Carroll et al., 1989; Todorow et al., 1989; Schroeder et al., 1990; Aigner et al., 1992; Bouquet et al., 1992). Although one laboratory has reported survival rates of up to 91%, fertilization up to 78%, and blastocyst formation as high as 54.4% (Carroll et al., 1993), it remains to be seen whether their results can be easily reproduced.

U.S. Pat. No. 5,985,538 issued Nov. 16, 1999 to Stachecki teaches a cryopreservation media containing less than 50 mM sodium ions, at least 100 mM choline salt, with an effective amount of a cryoprotectant. The basic medium on which Stachecki's cryopreservation medium is based employs a phosphate-buffered saline (PBS) solution. It is also known that a HEPES-buffered medium containing physiological levels of inorganic salts, a mixture of energy sources, including glucose, lactate, and pyruvate, as well as a range of amino acids is more likely to preserve oocytes and embryo viability after cryopreservation than one based on a phosphate-buffered solution (See, U.S. Pat. No. 5,716,847, issued Feb. 10, 1998, to Simmons et al.)

Thus, it would be a significant contribution to the art to provide a media for use in the cryopreservation of mammalian cells, including oocytes, wherein said cryopreservation media is sodium-depleted, does not employ a PBS buffer, employs a HEPES or MOPS buffer, contains less than 100 mM choline chloride, and wherein the cryopreserved mammalian cells, including oocytes, retain at least the amount of viability equivalent to embryo freezing, which is about 70-90% viability.

### SUMMARY OF THE INVENTION

The present invention provides a sodium-depleted composition that does not employ PBS as the buffer for the cryopreservation of mammalian cells, including oocytes.

The present invention further provides a method for using this composition in the cryopreservation of mammalian cells, including oocytes.

Still further provided are oocytes which have been cryopreserved using this composition.

### DETAILED DESCRIPTION OF THE INVENTION

The long term storage of mammalian cells that retain viability is of value, and has many potential uses, such as in research and in treatment. Additionally, long term storage of oocytes and/or ovaries is desirable for women undergoing chemo- or radio-therapy for the treatment of cancers, bone marrow transplantations, or other procedures that have the potential to leave the individual sterile. Timely prior long term storage of oocytes or ovaries well before perimenopause and menopause, or in the event of injury, infection or other means which would result in reduced fertility or loss of fertility, would insure fertility, even as a result of the aging process. This procedure would allow for the possibility of restored fertility, by a process including oocyte retrieval or surgical removal of a portion of the ovary or the whole ovary, cryopreservation in a reduced-sodium cryopreservation medium, followed by surgical implantation of the cryopreserved tissue or fertilized oocyte. As stated above, cryopreservation and transplantation of ovarian autografts, using simple embryo freezing protocols, have been successful in mice and sheep (Gosden et al., 1994; Gunasena et al., 1997). Storage of ovarian slices may be improved in a process employing the reduced sodium cryopreservation media according to the present invention, since a greater proportion of oocytes frozen in the present cryopreservation media survive and develop, compared to conventional sodium-based media. This same type of technique may also be used for the cryopreservation of other tissues and organs, for later transplantation or implantation.

To significantly improve oocyte freezing and enable its routine use, the present invention provides a cryopreservation media which, in part, alleviates the damaging effects of sodium transport across cell membranes and/or sodium loading of the cell. Because of the disruptive effects of ions, particularly sodium ions during cell freezing, the present invention seeks to substitute another ion. The preferred major cation in the cell medium according to the present invention is choline. Choline is the common name for 2-trimethyl amino 1-ethanol, a quaternary amine, which is accompanied by a suitable counterion. Toner et al. (1993) investigating whether cryoprotectants were absolutely necessary for cryopreservation, reported that mouse zygotes lysed upon warming to room temperature after being cooled to −40° C. in a hypertonic sodium chloride (NaCl)-supplemented phosphate buffered saline (PBS) solution lacking cryoprotectants. In contrast, the majority of zygotes cooled to −40° C. in a hypertonic choline chloride (ChCl)-supplemented phosphate buffered saline (PBS) solution lacking cryoprotectants, remained intact after warming to room temperature. While the membranes remained intact, these zygotes did not develop normally, indicating that while the ChCl prevented lysis, it was not sufficient to promote development. This study did not seek to address whether the ChCl might be used to replace NaCl, or the effect of a mixture of ChCl and cryoprotectant. However, it is believed that the choline anion used as the substitute for sodium results in the depletion of sodium.

The present invention provides an improved culture method and cryopreservation medium for the storage of oocytes which allows an oocyte to remain viable through a freeze/thaw cycle, as well as providing improved cryopreservation for other cell types. The cryopreservation solution according to the present invention employs an improved base-medium composition used for cryopreserving oocytes.

The composition of the present invention may also be employed as a medium for storing cells having a low sodium concentration. The medium may include, for example, a cryoprotectant, such as 1,2-propanediol, present in an amount of from about 100 mM to about 2500 mM, and preferably about 1500 mM, a natural or artificial serum protein, for example fetal bovine serum, for example, present in an amount of about 5-20%, and may be selected from one or more of the group consisting of fetal bovine serum, newborn calf serum, bovine serum albumin, human serum albumin, human cord serum, and plasminate, and consisting of a HEPES or MOPS buffered physiological solution. HEPES is N-2-hydroxyethylpiperazine-N′-2-ethanesulphonic acid. MOPS is 4-Morpholinepropanesulfonic acid. The concentration of the HEPES or MOPS employed in the buffering solutions is from about 10 to about 25 mM, with the preferred concentration being about 21 mM. The cryoprotectant is preferably present in an amount effective to inhibit crystallization of water when frozen, and may be selected from one or more of the group consisting of 1,2 propanediol, dimethyl sulfoxide, glycerol, and ethylene glycol, and for example, consists of 100 to about 2500 mM 1,2 propanediol.

Unless otherwise specified, the term “wt %,” as employed throughout the specification and claims, refers to weight percentage of the total composition calculated on a w/w basis in the aqueous or liquid phase.

As used with reference to cell freezing, the term “cryoprotectant” refers to a molecule that protects cells during a freeze-thaw cycle, promoting survival and retention of viability. The benefits derived from cryoprotectants are related to 1) their concentration, 2) exposure times, and 3) the temperature at which they are added to oocytes.

The term “osmolality,” as used herein, is a measure of the osmotic pressure of dissolved solute particles in an aqueous solution (e.g., an extender). The solute particles include both ions and non-ionized molecules. Osmolality is expressed as the concentration of osmotically active particles (i.e., osmoles) dissolved in 1 kg of water.

The cryopreservation media of the present invention is sodium depleted, and thus relies on the elimination of NaCl to prevent membrane damage, whereas membrane lysis frequently occurred in cryopreservation medium that was supplemented with NaCl. The present invention therefore eliminates most of the sodium from the cryopreservation medium composition, and replaces it with choline or another suitable cationic species. Choline is the common name for 2-trimethyl amino 1-ethanol, a quaternary amine, and therefore is accompanied by a counterion. Choline is involved with membrane chemistry (e.g., phosphatidyl choline) and intercellular (neurotransmitter acetyl choline) communication. While a direct relation between the cryopreservation solution according to the present invention and these biochemical pathways is not yet understood, the cryopreservation effect may be related. Therefore, compounds which interact or substitute for choline in these pathways may also be useful according to the present invention. The relatively large effective (hydrated) ionic size and low diffusion rate through the cell membrane of choline are believed to be important characteristics. Therefore, other quaternary amines or molecules positively charged at physiological pH may also be useful as cryopreservation solution components.

The cryopreservation media according to the present invention is sodium depleted, and therefore preferably has less than 7 mM sodium and preferably 1-2 mM sodium.

The present invention therefore provides a cryopreservation solution having a low sodium concentration and providing choline as a cation species. While one embodiment according to the present invention provides low sodium concentrations, it is also possible to replace nearly all of the sodium, for example, by substituting potassium bicarbonate for sodium bicarbonate (preferred concentration of 4 mM, with a range from 3.0 to 5.0 mM), pyruvic acid for sodium pyruvate (preferred concentration 0.33 mM, with a range from about 0.25 mM to about 0.40 mM), the dipotassium salt of EDTA for the tetrasodim salt of EDTA (with a preferred concentration of 0.01 mM, with a range of from 0.001 to 0.1 mM) the acidic form of phenol red for the sodium salt of phenol red (preferred concentration of 0.008 mM, with a range of 0.0001 to 0.01 mM), and potassium hydroxide for sodium hydroxide used to adjust the pH of the solution to a preferred range value of pH 7.40 with a range of 7.30 to 7.50. This would leave only trace amounts of sodium ions found in the protein solution added to the medium and water used to make the medium.

Choline chloride (ChCl) concentrations provided in the present composition are suitably from about 85 to about 99 mM. Particularly preferred for the practice of the present invention is a cryopreservation media composition containing choline chloride concentrations of about 90.6 mM.

The concentration of ChCl in the cryopreservation solution composition may also be increased from about 90.6 mM to about 99 mM. This would in effect dehydrate the preserved cell more and help reduce the chance of IIF from occurring.

The cryopreservation media composition may also be modified or altered, for example, by the addition of hyaluronic acid, or the like. Typically, the hyaluronic acid is present in a range of from about 0.1 mg/ml to about 1.0 mg/ml based on the total volume of the cryopreservation media.

The ChCl-based culture medium may also be used in other circumstances not involving cryopreservation, where a reduced sodium medium is desired.

Most of the mortality occurring when oocytes are cryopreserved, such as mouse oocytes, are related to cellular disruption by sodium ions. The conventional sodium-based freezing medium was found to be detrimental to oocyte survival, fertilization, and subsequent development in part because of sodium toxicity. According to the present invention, the nonpermeable ionic molecule choline, can substitute for sodium thereby maintaining membrane integrity after oocytes are thawed. Furthermore, the majority of oocytes frozen in ChCl-based medium survive cryopreservation, fertilize, and cleave to peri-implantation stages in vitro. In mouse oocytes, a high degree of survival (75%) was observed, as well as fertilization, and development for mouse oocytes using a simple freezing protocol. Following embryo transfer to pseudopregnant murine dams, blastocysts derived from cryopreserved oocytes are capable of implanting into a receptive uterus and developing to term. (Stacheki et al, 1998). These data represent a highly significant improvement in the ability to cryopreserve mouse oocytes. This advancement was made possible by an improvement in cryopreservation medium composition, specifically the removal of NaCl and its replacement with ChCl and basing the cryopreservtion medium on a HEPES or MOPS-buffered physiological solution rather than on a modified phosphate-buffered solution. This technology has allowed mouse oocytes to be easily and reproducibly frozen and has applications for the storage of oocytes from other species, as well as other cells including embryos, sperm, erythrocytes, and tissues. A cryopreservation solution according to the present invention therefore provides an environment which assures a high degree of survival, fertilization, and development for oocytes.

The cryopreservation media and methods of the present invention are capable of preserving oocytes such that a high percentage will survive thawing, and demonstrate high cleavage rates upon activation. Such oocytes are deemed to be viable.

The following Examples are provided to further demonstrate the present invention.

### EXAMPLE 1

### Preparation of Cryopreservation Media

A cryopreservation solution was prepared with the composition shown in Table 1 below, then diluted with distilled water to make 1 liter total volume.

TABLE 1
FORMULATION FOR SODIUM DEPLETED SAGE
FREEZING MEDIUM

| | | | | Suggested | |
|---|---|---|---|---|---|
| | | | milli mole | range | |
| Ingredient | g/liter | | mM | mM | |
| Choline chloride | 12.650 | | 90.6 | 85-99 | |
| Potassium chloride | 0.350 | | 4.7 | 4.0-5.0 | |
| Magnesium sulfate, | 0.050 | | 0.2 | 0.05-2.0 | |
| heptahydrate | | | | | |
| Potassium phosphate, | 0.0014 | | 0.01 | .001-0.05 | |
| monobasic, anhy | | | | | |
| Potassium bicarbonate | 0.400 | | 4.0 | 3.0-5.0 | |
| HEPES, free acid | 5.004 | | 21.0 | 18.0-24.0 | |
| Glucose, D-(+) | 0.500 | | 2.78 | 2.0-3.5 | |
| Pyruvic acid | 0.029 | | 0.33 | 0.25-0.40 | |
| Calcium lactate, | 0.629 | | 2.04 | 1.5-2.5 | |
| pentahydrate | | | | | |
| Alanyl-glutamine | 0.217 | | 1.0 | 0.01-2.0 | |
| L-Asparagine | 0.013 | | 0.1 | 0.01-0.5 | |
| L-Aspartic acid | 0.013 | | 0.1 | 0.01-0.5 | |
| Glycine | 0.008 | | 0.1 | 0.01-0.5 | |
| L-Proline | 0.012 | | 0.1 | 0.01-0.5 | |
| L-Serine | 0.011 | | 0.1 | 0.01-0.5 | |
| EDTA, dipotassium salt, | 0.004 | | 0.01 | 0.001-0.1 | |
| dihydrate | | | | | |
| Phenol red | 0.003 | | 0.008 | 0.0001-0.01 | |
| Human albumin | 12.000 | g/L | | 10-20 | g/L |
| Gentamicin | 0.010 | | | 0.001-0.02 | g/L |
| 1,2-Propanediol | 114.150 | | 1,500 | 200-2,500 | |
| Sucrose | 102.69 | | 300 | 50-1000 | |

All procedures for formulating the medium are carried out at room temperature. With mixing, add the weighed quantities of each ingredient to a bulk compounding vessel in the order listed in Table 1. Allow each ingredient to dissolve before adding the next ingredient. After addition of last ingredient, allow the bulk to mix with stirring.

Obtain a 5 mL sample of the bulk solution and test for pH. Adjust the pH to 7.35 (+/−0.05) with 10 N KOH.

Obtain a sample of the bulk solution and test for final pH, osmolality, color and clarity.

Bulk solution may be stored at 2-8° C. in a closed container for up to 24 hours prior to filling. If bulk solution is held in storage, test pH prior to filling to insure that the pH is 7.3-7.4 at 30-35° C.

The solution is then filtered through a 0.2 um pore sized filter and aseptically filled into suitably sized containers to which sterile closures are applied. The containers are then labeled with a label indicating the name of the product, volume of medium within the container, lot number of the product and its date of expiration, assuming an expiration date of one year from the date of production.

### EXAMPLE 2

### Collection and Culture of Mouse Oocytes

Follicular activity may be stimulated in 4-6 week old female C57BL/6 X C3HF1(B6C3 F1) mice (Charles River Laboratories, Wilmington, Mass.) by intraperitoneal injection of 10 IU equine chorionic gonadotropin (Gestyl Professional Compounding Centers of America, Houston, Tex.), followed 48 h later with 10 IU hCG (Sigma Chemical Company, St. Louis, Mo.). Cumulus masses were collected from oviducts 14 h post-hCG and treated with 200 U/ml hyaluronidase (Sigma) for 10 min to remove cumulus cells. The oocytes were washed in Quinn's Sperm Washing Medium (Sage In-Vitro Fertilization, Inc, Trumbull, Conn.) and held at RT until cryopreservation or fertilization. All cell culture was carried out in a 5% CO₂:5% O₂:90% N₂ in an incubator at 37° C., using microdrops (10 microliters) of Quinn's Advantage Fertilization Medium (Sage) in 35 mm×10 mm Cell Culture Dishes (Coming: VWR Scientific, Piscataway, N.J.) flooded with Oil for Tissure Culture (Sage).

Mouse Oocyte Freezing and Thawing:

Oocytes that were translucent, round, having extruded the first polar body, and appeared normal, were selected for cryopreservation. Several of these oocytes were set aside to be used as nonfrozen controls. Oocytes were cryopreserved in a newly-formulated modified HEPES-HTF medium according to the present invention, having a composition as shown in Table 1. Oocytes were pre-equilibrated at 23° C. in the Cryopreservation solution according to the present invention containing 1.5 M cryoprotectant for 10 min and then transferred to Cryopreservation solution according to the present invention containing 1.5 M cryoprotectant and 0.3 M sucrose for 20 min. During this time the oocytes were loaded into 0.25 ml French straws (Agtech, Inc, Manhatten, Kans.) and heat sealed at the open, non-plugged end. The straws were placed in a Freeze Control Preprogrammed Temperature Controlled freezer (Biogenics, Napa, Calif.) which had been precooled to −8° C., seeded using forceps cooled in LN₂, held at −8° C. for 10 min, and cooled at a rate of −0.3° C./min to −35° C. before plunging into LN₂. Oocytes were thawed by exposing the straw to air for 10-30 sec before immersing in a 30° C. water bath for an additional 10 sec. The oocytes were expelled from the straws into a solution according to the present invention containing 0.5 M sucrose at 23° C. and held in this solution for 10 minutes, then transferred to a solution according to the present invention containing 0.2M sucrose at 23° C. for another 10 minutes, and then rinsed in a solution according to the present invention containing no sucrose. All these freezing and thawing solutions for mouse oocyte cryopreservation contained 20% (v/v) Fetal Bovine Serum (Gemini Bio-Products, Inc., Calabasas, Calif.).

Results on freezing mouse oocytes with cyropreservation medium: Effect of medium type on survival and fertilization of mouse oocytes after freezing and thawing:

| | Sage Freezing Medium | Stachecki |
|---|---|---|
| | (as in Table 1) | Freezing Medium |
| Number frozen | 29 | 29 |
| Survival (%) | 29 (100) | 21 (72) |
| Number fertilized | 25 | 15 |
| % of surviving oocytes | 86% | 71% |
| % of frozen oocytes | 86% | 52% |

The proportion of frozen oocytes that were fertilized was significantly higher using the Sage Freezing Medium compared to the Stachecki medium.

### EXAMPLE 3

### Human Oocyte Cryopreservation

Human oocytes were cryopreserved using both the Sage medium and the Stachecki medium.

Sodium-depleted media for egg freezing in human. Note that these data have cycles in which either sodium-depleted PBS or Sage Freezing Medium as the base medium were used for freezing.
# of thaw cycles conducted: 31
# of patients with thaw cycles conducted: 28
# eggs thawed: 189
# eggs surviving thawing: 132 (70% of eggs thawed)
# eggs with ICSI: 130
# damaged post-ICSI: 26 (20% of injected oocytes)
# fertilized: 79 (60% of injected oocytes)
# embryo transfer procedures: 5 (note there were 6 cycles that did not get transferred; 2 due to fertilization failure and 4 because of embryonic arrest prior to ET)
# clinical pregnancies (one or more gestational sacs): 9
clinical pregnancy rate per thaw cycle=9/31=29%
clinical pregnancy rate per patient=9/28=32.1%
clinical pregnancy rate per transfer=9/25=36%
# miscarriages: 2
# ongoing pregnancies: 2
# deliveries: 5
# singleton: 3
# twin: 2 (See, Materials and Methods in Human Reproduction, Vol. 18, No. 6, 1250-1255, June 2003, the disclosure of which is herein incorporated by reference).

### EXAMPLE 4

### Mouse Oocyte Cryopreservation

Media based on HEPES-HTF and PBS were compared for their effectiveness to cryopreserve mouse oocytes.

A 2×2 factorial experiment based on a modified HEPES-HTF and a PBS-based medium, with or without sodium-depleted formulations was performed. Freshly collected oocytes from superovulated females were pooled, divided into four equal groups (29-38/group) and cryopreserved in 0.5 mL straws using a standard slow-cooling procedure. Nine to 147 (av. 61) days later, the oocytes were thawed, inseminated in vitro and resulting embryos cultured to the blastocyst stage. The experiment was replicated four times with a total of 136 oocytes in each treatment. Recovery, survival, fertilization and development rates between the four treatments were analyzed by-square.

One third of all oocytes frozen in HEPES-HTF (HTF is human tubal fluid) survived, were fertilized and developed to blastocysts. This was more than double the proportion in PBS-based media. The blastocyst development rates with the sodium-depleted HEPES-HTF based medium (Sage Freezing Medium) in the present study were 22% higher (39% v. 32%) than those reported by Stachecki et al (1998) using PBS-based sodium-depleted medium. There were no significant differences between sodium-depleted and regular media in either of the two different formula based media. Eighty five percent of freshly collected control oocytes developed to blastocysts.

Conclusions: A medium based on a modified HEPES-HTF formulation was superior to one based on PBS for the cryopreservation of mouse oocytes. This HEPES-HTF medium has been used for human oocytes with higher pregnancy rates than those obtained with PBS-based medium (Boldt, unpublished; see Example 5 below). The mouse model appears to emulate the results obtained in humans.

### EXAMPLE 5

### Human Oocyte Cryopreservation as an Alternative to Embryo Cryopreservation in Art Cycles

OBJECTIVE: To examine the experience with frozen egg-embryo transfer (FEET) and to compare results from FEET with frozen embryo transfer (FET).

SETTING: Hospital based IVF program.

PATIENTS: 24 couples undergoing IVF-ET cycles that had oocytes frozen and thawed, using a sodium depleted freezing solution. In addition, we studied 62 couples in which embryos were frozen on day 3 post-retrieval and were subsequently thawed and transferred.

INTERVENTIONS: Patients for oocyte cryopreservation had standard IVF-ET therapy, and had supernumerary oocytes frozen and thawed using either sodium-depleted phosphate buffered saline (PBS) or modified HTF (mHTF) based freezing medium in conjunction with a slow freeze-rapid thaw procedure. Upon thawing, surviving oocytes were inseminated by ICSI, and embryos were then transferred using standard embryo transfer procedures. Thawed embryo transfer was accomplished using standard thawing and transfer procedures.

MAIN OUTCOME MEASURES: For FEET, survival rates, fertilization rates, pregnancy rates, and implantation rates per egg thawed and per embryo transferred were calculated. Survival rates, pregnancy rates, and implantation rates per embryo for oocyte freeze-thaw cycles were compared to the same parameters calculated for FET cycles.

RESULTS: In 30 FEET cycles, we obtained an overall 56.4% survival rate post-thaw and a 65.9% fertilization rate following ICSI. With PBS based media survival and fertilization rates were 47.2% and 58.8% and with mHTF they were 60.8% and 68.5%. In 25 ET cycles from FEET, we obtained 9 pregnancies and 6 live born infants (30% pregnancy rate per thaw and 36% pregnancy rate per ET), with a 4.0% implantation rate per egg thawed and a 13.4% pregnancy rate per embryo transferred. Comparing our overall FEET results (including cycles reported on in 2003) to those obtained from FET, there was no difference in survival, pregnancy, and implantation rates.

CONCLUSIONS: FEET demonstrated that good survival and pregnancy rates can be attained with oocyte cryopreservation, and can offer pregnancy and implantation rates comparable to FET.

Introduction:

The ability to successfully freeze and thaw human oocytes has a number of implications for reproductive medicine. There are several clinical scenarios where the ability to store oocytes would be of benefit. These include:

Patients undergoing ART therapies that do not wish to freeze embryos for religious or other reasons. By freezing oocytes such patients could have several attempts at pregnancy from a single egg retrieval cycle. This procedure also does not involve the legal and ethical issues around freezng and storing embryos.
1. Patient with medical conditions such as cancer requiring treatments that may put them at risk for permanent loss of ovarian function.
2. Patients that are concerned with age-associated decline in fertility that would like to freeze and store eggs at an earlier age for use later in life.
3. Donor egg therapy in which frozen eggs would allow for quarantine and testing of donors for infectious diseases prior to use.
4. Countries in which embryo freezing is banned.

The program has previously reported on our initial work with oocyte cryopreservation and thawing in women undergoing ART therapy, using a sodium-depleted phosphate buffered saline medium as the base medium for the freezing solution. In the present communication, further data is provided on the efficacy of oocyte cryopreservation and compare two different sodium-depleted media for cryopreservation. Pregnancy rates were compared with frozen oocyte vs. frozen embryo transfer to determine the efficacy of oocyte cryopreservation in the program.

Methods

The data presented were obtained from a series of 30 frozen egg-embryo transfer (FEET) cycles in 24 patients conducted from January 2002-October 2004. All thaw cycles done during this time are included in the analysis. Each of the patients included in the analysis had undergone a fresh ART cycle, and had declined embryo freezing because of religious or ethical concerns. In such cases, our protocol was to inseminate only a number of eggs equivalent to the number of embryos that the patient would wish transferred. During the period of the study, egg freezing was under an investigational protocol approved by the Community Hospital Institutional Review Board for research projects. All patients signed an informed consent attesting to the investigational nature of oocyte cryopreservation before eggs were frozen. There were no donor egg cycles included in this set of patients.

Patients were stimulated for IVF using standard ART stimulation protocols. Patients were either down regulated with luteal-phase leuprolide acetate suppression, or were placed on GnRH antagonist suppression when at least 4 follicles reached a maximum diameter of 12 mm. Pure FSH (either Gonal F or Follistim) was used for stimulation, and patients received hCG (10000 IU IM) when at least 4 follicles were >=18 mm maximum diameter. Transvaginal oocyte retrievals were carried out 35-36 hours after hCG administration. Eggs were examined for the presence of a first polar body, and several eggs (3-5 depending on patient age) were set aside for insemination in a “fresh” IVF cycle. The remaining eggs were exposed to hyaluronidase (80 IU/ml) for approximately 30-60 seconds; during this time cumulus masses were aspirated through a pipette to remove excess cumulus cells. The eggs were then transferred to fresh culture media, and the adhering corona radiate cells removed by aspiration through narrow bore micropipettes. Mature eggs with an extruded first polar body were selected for cryopreservation.

Two different media were used for oocyte cryopreservation in the cycles reported herein. For 8 FEET cycles, sodium depleted phosphate buffered saline (PBS) supplemented with 20% synthetic serum substitute and containing 1.5M propanediol/0.2 M sucrose was used for freezing. The PBS media was made in the laboratory using cell culture grade reagents purchased from Sigma (St. Louis, Mo.). For 22 FEET cycles, a sodium-depleted modified human tubal fluid medium supplemented with 12 mg/ml human serum albumin and containing 1.5M propanediol and 0.3M sucrose was used for cryopreservation. The mHTF based freeze media was manufactured by SAGE In Vitro Fertilization Inc., a Cooper Surgical Company, Trumbull, Conn.

For cryopreservation, eggs were incubated in 1.0 ml of freezing solution for 20 minutes at ambient temperature (22-24° C.). After the 20 min exposure to cryoprotectant, eggs were loaded into freezing vials (Nunc) containing 0.5 ml of freezing solution and were then loaded into a Planar freezer set at 22° C. The vials were cooled at −2° C./min to −6° C., held at −6° C. for 5 min, then seeded by touching the outside of the vial at the fluid meniscus with a pair of metal forceps cooled in liquid nitrogen. The vials were then held for an additional 10 min at −6° C., then cooled at −0.3° C./min to −35° C., at which time the vials were plunged into liquid nitrogen and then stored in liquid nitrogen tanks until thawed.

In preparation for thawing patients were given oral estradiol, 2 mg./day from cycle day (CD) 5 to CD 8, 4 mg./day CD 8 to CD12, and 6 mg/day CD 12 to CD 18. On CD 18 a vaginal ultrasound was done to assess endometrial thickness. If the endometrium was not =>9 mm. the patient was continued on 6 mg./day and reassessed every other day until the desired thickness of =>9 mm. was obtained. Once the desired endometrial thickness was obtained the dosage of estradiol was dropped to 4 mg./day until either a negative BhCG was present at the end of the FEET cycle or, if the patient was found to be pregnant, the dosage was continued. When the endometrium reached >=9 mm the patient began taking progesterone in oil, 25 mg IM, and eggs were thawed on the first day of progesterone administration, with ET on day 4 of progesterone. Progesterone was increased to 50 mg on the second day, and then increased to 100 mg on the third day; patients were continued on 100 mg IM progesterone in oil for 12 days until a pregnancy test was done. Pregnant patients were continued on oral estradiol 4 mg./day and progesterone in oil 100 mg. IM/day. Serum estradiol and progesterone levels were obtained every two weeks and the dosage of each adjusted in an effort to keep the estradiol levels near or above 50 pg/ml. and the progesterone levels near or above 50 ng./ml. All patients were kept on this protocol until 10 weeks gestation. They were then slowly weaned off the supplemental estradiol and progesterone for the next two weeks and transferred to the referring obstetrician after 12 weeks.

For thawing, vials were removed from liquid nitrogen storage, and the tops loosened to vent any nitrogen that may have entered the vial during storage. The vials were then thawed in a 32° C. water bath until all ice crystals disappeared; thawing time was approximately 1-1.5 minutes. The contents of the vial were then pipetted onto the lid of a Falcon 3003 or 3037 dish, and the eggs identified and moved to a #3037 dish containing 1.0 ml of 0.5M sucrose. After 10 min at room temperature the eggs were transferred to another 3037 dish containing 1 ml of 0.2 M sucrose, and held for another 10 min at room temperature. The sucrose solutions were made with either sodium depleted PBS or sodium-depleted SAGE modified HTF. After 10 min in 0.2M sucrose, the eggs were washed in Quinn's fertilization media (SAGE Biopharma) with 0.5% HSA, and cultured in 50 ul drops of this media under mineral oil for 2-5 hours until insemination. All inseminations were done by ICSI to avoid potential fertilization problems associated with premature cortical granule release. Because patients were not willing to have embryo freezing performed, in general thawing was performed until there were 3-4 eggs that had survived both the thawing and ICSI procedures because this was the maximum number of embryos advised for transfer. In one case, a patient with 5 eggs requested that all eggs be thawed and that any embryos be transferred regardless of number; in this case 5/5 eggs survived thawing and fertilized after ICSI, and a singleton gestation with a live born infant was obtained after transfer of 5 embryos.

Eggs were examined for fertilization at 16-20 hours post-ICSI, and eggs with two pronuclei cultured in 50 μl drops of Quinn's cleavage media (SAGE In Vitro Fertilization, Inc.) supplemented with 10% SPS. Embryo transfers were carried out on day 3 post-thaw, with all embryos hatched using acid Tyrode's a minimum of 1 hour before ET. All ET's were done under ultrasound guidance using Wallace catheters.

Data comparing egg freezing to embryo freezing was obtained by comparing results from egg freeze/thaw cycles to those obtained with day 3 embryo freezing and thawing. We chose day 3 embryo freezing because the vast majority (over 95%) of our embryo freeze/thaw cycles involved day 3 frozen embryos. For embryo freezing data, an embryo was deemed to have survived if 50% or more of the blastomeres remained intact after thawing. Patients were placed on estrdiol/progesterone replacement regimens as for egg thaw cycles, and the embryos were thawed and transferred on day 4 of progesterone administration. Comparisons were made with respect to survival rates post thaw, pregnancy rates, and implantation rates per embryo thawed and/or transferred. We were unable to compare pregnancy outcomes because a number of FET patients were lost to follow-up.

Data on survival rates, fertilization rates, pregnancy rates, and implantation rates wee analyzed by chi square test, with significant differences defined at the p<0.05 level.

Results

The results from egg freezing using either sodium depleted PBS or sodium depleted modified HTF as base medium are shown in Table 1. Overall there were 30 thaw cycles conducted in 25 women from January 2002-October 2004, resulting in 25 embryo transfer procedures. Of the 5 cycles that did not proceed to embryo transfer, one had none of 3 available eggs survive the thaw. Of interest, this patient had 4 eggs thawed from the same cohort of frozen eggs in a prior thaw cycle, all of which survived thawing, and generated a pregnancy that miscarried at 9 weeks gestation. There were 2 cycles in which either no eggs fertilized or there was no normal fertilization (defined as the presence of 2 pronuclei) obtained. The remaining 2 non-transfer cycles resulted from cycles in which embryo development arrested prior to the time of transfer.

An average of 9 oocytes were thawed in the PBS group vs 7.0 in the mHTF group. The survival rates with FEET in the 8 cycles using PBS in the freezing solution was lower (47.2%) than observed with mHTF (60.8%), but did not reach statistical significance. Fertilization rates were also slightly lower in the PBS group (58.8%) vs. the mHTF group (68.5%), with the difference not reaching statistical significance. This was likely due to a higher damage rate post-ICSI with the PBS group (26.5%) as compared to the mHTF group (12.0%).

There were 9 pregnancies established overall in these patients, yielding a 30.0% pregnancy rate per thaw attempt, and a 36% pregnancy rate per patient or per ET. Of these, there was one biochemical pregnancy with no discernable gestational sac; this patient terminated use of supplemental progesterone for unknown reasons two days after the positive hCG was detected. There was one pregnancy with a gestational sac but no fetal heart beat, two pregnancies with a single and a twin fetal heart that miscarried, one twin fetal heart gestation in which one of the fetuses underwent intrauterine demise, and one neonatal demise at 22 weeks when the patient had to be delivered secondary to severe pre-eclampsia. Overall there were a total of 9 documented fetal heart implantation sites identified in the 9 pregnancies obtained, yielding an implantation rate per embryo transferred of 13.4%, and an implantation rate per egg thawed of 4.0%. For the 8 PBS cycles, there was only one pregnancy with a gestational sac but no fetal heart beat. For mHTF cycles, there were 8 pregnancies obtained in 22 thaw and 18 transfer cycles, yielding a 36.3% pregnancy rate per thaw attempt and a 44.4% pregnancy rate per thaw. The implantation rate per egg thawed and per embryo transferred for the mHTF cycles was 5.9% and 17.6%, respectively.

To compare the efficiency of egg freezing to embryo freezing, we compared clinical results from egg freeze/thaw cycles to those obtained from day 3 embryo freeze/thaw cycles. For this comparison, we included data from 16 thaw cycles in 11 patients that had been reported on previously. The results are shown in Table 3. The survival rates of eggs vs. embryos post-thaw were statistically identical, as were both pregnancy rates per transfer and implantation rates per embryo transferred.

Discussion

The experience with oocyte cryopreservation indicates that oocyte freezing/thawing can be routinely performed, yielding survival and pregnancy rates comparable to those obtained with embryo freezing. This method employs use of a sodium-depleted base medium, which in animal models has been shown to improve post-thaw survival rates. In addition, alterations in the dehydration time by extending time in cryoprotectant to 20 minutes prior to freezing, raising the seeding temperature, and increasing sucrose concentration in the freeing medium may also help post-thaw survival. The strategy provides a greater than 50% survival rate post-thaw, with a pregnancy rate per thaw of almost 30% and a pregnancy rate per transfer of approximately 36%. It is important to note that these results may represent a lower, rather than upper, limit of what might be gained from egg freezing. Because patients in this series would not agree to embryo freezing, a relatively small number of eggs were thawed and inseminated in each cycle. If more eggs were thawed and fertilized, pregnancy rates might be expected to increase due to enhanced capability for embryo selection prior to transfer.

The data suggest that mHTF may be a better base media for oocyte freezing than PBS, as survival, fertilization, and pregnancy rates were higher in cycles where mHTF was used to prepare the freeze/thaw solutions. The number of cycles reported in this paper for PBS are somewhat low, however. If we take into account our previous series of 16 thaw cycles where PBS was used for preparation of the freeze/thaw solution, our overall results with PBS provided a 62% survival rate, a 55% fertilization rate, a pregnancy rate of 20.8% per thaw and 27.8% per ET, and implantation rates per egg thawed and per embryo transferred of 3.1% and 11.4%. Each of these is less than the rates obtained with mHTF cycles, as seen in Table 1. Although none of the differences observed were found to be statistically significant based on the small sample size, the trend towards higher results for all these parameters with the mHTF-based cryopreservative suggests it is preferable for oocyte cryopreservation.

For egg freezing to be performed on a routine basis by all centers, methods must be developed that: 1. are reproducible and effective; 2. are easily assimilated into the reproductive laboratory, and 3. yield pregnancy, delivery, and birth defect rates consistent with existing technology. With respect to the reproducibility and effectiveness of egg freezing, the methods used currently for egg freezing fall into two general categories: slow freezing methods and vitrification strategies. As to the former, a number of laboratories have now reported successful thawing results, with pregnancies and live born infants, using adaptations of slow freeze/thaw approaches. The different groups reporting on slow freezing for egg cryopreservation all have somewhat different strategies. For example, a sodium-depleted media has been employed for egg freezing, and others employ equilibration of the oocyte in cryoprotective media at 37° C. as part of the freezing strategy. Several groups have used elevated extracellular sucrose in their cryoprotective solutions. Ultimately, such approaches represent somewhat minor variations to techniques for embryo freezing that have been used for decades, and as such can be readily incorporated into any IVF laboratory.

Vitrification, involving the use of high concentrations of cryoprotectants to avoid intracellular ice formation, has also been used successfully for oocyte preservation with several births reported. There has been a lack of uniformity in vitrification strategies. Taken together, the data as well as reports from a number of centers now indicate that egg freezing can be done reproducibly and with high efficiency, with further work required to evaluate different methods to determine whether any one approach is most successful.

The final requirement for routine use, i.e, pregnancy, delivery and birth defect data similar to existing technology, involves several issues. First, egg freezing should provide equivalent pregnancy rates to alternative technologies, such as embryo freezing. The data, comparing oocyte thawing to thawing of day 3 cryopreserved embryos, indicates that identical implantation and pregnancy rates can be obtained. Similarly, Yang et al. have indicated identical pregnancy rates in frozen egg vs. frozen embryo cycles, and Porcu et al. have shown no difference in pregnancy rates between fresh and frozen oocyte cycles. In contrast, Borini et al. have shown that their implantation rate per inseminated egg was 3.9% in frozen embryo cycles compared to 2.2% for frozen egg cycles suggesting egg freezing is less efficacious. Thus, while these studies indicate that egg freezing can be done with efficiencies approaching or even equaling that of embryo freezing, further work is required to make egg freezing even more efficacious.

Whether egg freezing would result in a higher incidence of pregnancy abnormalities, birth defects, chromosomal anomalies, or long term health effects to the offspring remains open to question. The limited data indicates that, of the 13 pregnancies established, 4 were lost, yielding a 30% loss rate that is higher than the approximate 18% loss rate we observe with fresh IVF cycles in our program. Other small series have suggested a loss rate of 20-50%. The cooling and eventual freezing of the egg during cryopreservation causes loss of the egg's meiotic spindle due to depolymerization of spindle microtubules, and as such might cause an increased risk of aneuploidy after thawing and fertilization. Several studies have demonstrated, however, that upon thawing and rewarming a high percentage of eggs reform their spindle apparatus with no loss of chromosomes. While these data are reassuring, care should be taken to accumulate the worldwide data on survival, pregnancy, and birth data to determine the overall benefit of

### REFERENCES

Agca, Y., Liu, J., Rutledge, J. J., Critser, E. S. and Critser, J. K. (2000) Effect of osmotic stress on the developmental competence of germinal vesicle and metaphase II stage bo me cumulus oocyte complexes and its relevance to cryopreservation. Mol. Reprod. Dcv., 5, 212-219. [[
Al-Hasani, S., Diedrich, K., Van der Ven, H., Reineke, A., Hartje, M. and Krebs, D. (1 87) Cryopreservation of human oocytes. Hum. Reprod., 2, 695-700.
Boldt J, Cline D, McLaughlin D. Human oocyte cryopreservation as an adjunct to IVF-embryo transfer. Hum Reprod 2003; 18: 1250-55.
Borini A, Bonu M A, Coticchio G, Bianchi V, Cattol M, Flamigni C. Pregnancies and births after oocyte cryopreservation. Fertil Steril 2004; 82: 601-05.
Chen, C. (1988) Pregnancies after human oocyte cryopreservation. Ann. N.Y. Acaä. Sc 541, 41-549.
Chen, S. U., Lien, Y. R., Chao, KIT., Lu, H. F., Ho, H. N. and Yang, Y. S, (2001) Cryopruserviiticin of mature human oocytes by vitrification with ethylene glycol in straws. Fertil. SteriL, 74, 804-808.
Chung, H. M., Hong, S W., Lim, J. M., Lee, S. H., Cha, W. T., Ko, J. J., Han, S. Y., Choi, D. H. and Cha, K. Y. (2000) In vitro blastocyst formation of human oocytes obtained from unstimulated and stimulate cyc es Ster vitrification at various maturational stages. FertiL Steril., 73, 545.
Cobo, A., Rubio, C., Gerli, S., Ruiz, A., Pellicer, A. and Remohi, J. (2001) Use of fluo sce ice in situ hybridization to assess the chromosomal status of embryos obtained from cryopreserved oocytes. Fer/iL 5 er., 75, 354-360. [[
Coticchio, C., Garetti, S., Bonu, M. A. and Borini, A. (2001) Cryopreservation of humal.L oo(ytc:;. Hum. FerliL, 4, 152-157.
Fabbri R, Porcu E, Marsella T, Rocchetta G, Venturoli S, Flamigni C. Human oocyte cryopreservation: new perspectives regarding oocyte survival. Hum Reprod 2001; 16: 411-16.
Gook, D. A. and Edgar, D. H. (1999) Cryopreservation of the human female gamete: cui vent tad future issues. Hum. Reprod, 14, 2938-2940.
Gook, D. A., Osborn, S. M. and Johnston, W. I. (1993) Cryopreservation of mouse and h amm. Oc eytes using 1,2 propanediol and the configuration of the meiotic spindle. Hum. Reprod., 8, 11
Gook, D. A., Osborn, S. M., Boume, H. and Johnston, W. T. (1994) Fertilization of hum2 i 00:yt'ps following cryopreservation: normal karyotypes and absence of stray chromosomes. Hum. Repro 9, ;84. 691.
Fosas N, Marina F, Torres P J, Jove I, Martin P, Perez N, Arnedo N, Marina S. The births of five Spanish babies from cryopreserved donated oocytes. Hum reprod 2003: 18; 1417-21.
Gook, D. A., Schiewe, M., Osborn, S. M., Asch, R. H., Jansen, R. P. and Johnston, W. I. Intracytoplasmic sperm injection and embryo development of human oocytes cryopreserved using 1,2-propanediol. FIu'n. Reprod., 10, 2637-2641
Goud, A., Goud, P., Qian, C., Van der Elst, J., Van Made, G. and Dhont, M. (2000) Crypreservation of human germinal vesicle stage and in vitro matured MIT oocytes: influence of cryopreservatio mec ian the survival, fertilization, and early cleavage divisions. FertiL SteriL, 74, 487-494.
Hong, S W., Chung, H. M., Lim, J. M., Ko, J. J., Yoon, T. K., Yee, B. and Cha, K. Y. (199) Improved human oocyte development after vitrification: a comparison of thawing methods. Fert. Steril., 72, 1′ 2-1.6.
Katayama K P, Stehlik J, Kuwayama M, Kato O, Stehlik E. High survival rate of vitrified human oocytes results in clinical pregnany. Fertil Steril 2003; 80: 223-4
Kazem R, Thompson LA, Srikantharajah A, Laing MA, Hamilton M P, Templeton A. Cryopreservation of human oocytes and fertilization by two techniques: in vitro fertilization and intracytoplasmic sperm injection. Hum Reprod 1995; 10: 2650-54.
Kuleshova L, Gianaroli L, Magli C, Ferraretti A, Trounson A. Birth following vitrification of a small number of human oocytes: case report. Hum Reprod 1999; 14: 3077-9.
Kuleshova, L., Gianaroli, L., Magli, C., Ferraretti, A. and Trounson, A. (1999) Birth fc low ng vitrification of a small number of human oocytes: case report. Hum. Reprod., 14, 3077-3079.
Marina F, Marina S. Comments on oocyte cryopreservation. Reprod Biomed Online 2003; 6: 401-O₂.
Paynter, S. J. (2000) Current status of the cryopreservation of human unfertilized oocytes. B tm Reprod. Update, 6, 449-456
Pickering, S. J., Braude, P. R., Jonson, M. H., Cant, A. and Currie, J. (1990) Transient to room temperature can cause irreversible disruption of the meiotic spindle in the human oocyte. FertiL Steril., 54, 102 108.
Porcu, E., Fabbri, E., Seracchioli, R., Ciotti, P. M., Magrini, 0. and Flamigni, C. (19971 Bulb u a healthy female after intracytoplasmic sperm injection of cryopreserved human oocytes. Fertil. SteriL, :S, 77 [
Porcu, E., Fabbri, R., Damiano, G., Giunchi, S., Fartto, R., Ciotti, P. M. Venturoli, S. i F amigni, C. (2000) Clinical experience and applications of oocyte cryopreservation. Mol. Cell. Endocrino,., 1&, 31-37. [[
Porcu, E., Fabbri, R., Ciotti, P. M., Frau, F., D Cesare, R. and Venturoli, S. (2002) Oocyte and Embryo storage FertiL SteriL, 78 (Suppl.), Si 5 (abstract).
Quintans CJ, Donaldson M J, Bertolino M V, Pasqualini R S: Birth of two babies using oocytes that were cryopreserved in a choline based freezing medium. Hum reprod 2002; 17: 3149-52.
Rienzi L, Martinez F, Ubaldi F, Minasi M G, Iacobelli M, Tesarik J, Greco E. Polscope analysis of meiotic spindle changes in living metaphase II human oocytes during the freezing and thawing procedures. Hum Reprod 2004; 19:655-9.
Sathananthan, All., Trounson, A. and Freeman, L. (1987) Morphology and fertilization of frozen human oocytes. Gamete Res., 16, 343-354.
Sathananthan, A. H., Trounson, A., Freeman, L. and Brady, T. (1988) The effect of coding iur ian oocytes. Hum.Reprod., 3, 968-977. ct]
Stachecki, J. J., Cohen, J. and Willadsen, 5. (1998a) Detrimental effects of sodium during oocyte cryopreservation. Biol. Reprod., 59, 395-400.
Stachecki, J. J., Cohen, J. and Willadsen. S. M. (1998b) Cryopreservation of unfertilized mouse oocytes: the effect of replacing sodium with choline in the freezing medium. Cryobiology, 37, 346-3:54.
Stachecki, J. J., Cohen, J., Schimmel, T. and Willadsen, S. M. (2002) Fetal development of n xu!:e oocytes and zygotes cryopreserved in a non-conventional freezing medium. Cryobiology, 44, 5-13. Cro sR;.
Testart, J., Lasalle, B., Belaisch-Allart, J., Hazout, A., Forman, R., Rainhom, J. D. and rydinar. R. (1986) High pregnancy rate after early human embryo freezing. Fertil. SteriL, 46, 268-272.
Trad, F. S., Toner, M. and Biggers, J. D. (1998) Effects of cryoprotectants and ice-seeding temperture on intracellular freezing and survival of human oocytes. Hum. Reprod, 14, 1569-1577.
Tucker, M. J., Wright, G., Morton, P. C. and Massey, J. B. (1998) Birth after cryopreservation. of immature oocytes with subsequent in vitro maturation. FertiL Steril., 70, 578-579.
Van Blerkom, J. and Davis, P. W. (1994) Cytogenetic, cellular, and developmental con equ rLcc of cryopreservation of immature and mature human oocytes. Microsc. Res. Tech., 27, 165-193.
Van Uem, J. F. H. M., Siebzehrubl, E. R., Schuh, B., Koch, R., Trotnow, S. and Lang N. 198X Birth after cryopreservation of unfertilized oocytes. Lancet, 752-753.
Wang W H, Meng L, Hackett R J, Odenbourg R, Keefe D L. Limited recovery of meiotic spindles in living human oocytes after cooling-rewarming observed using polarized light microscopy. Hum reprod 2001; 16: 2374-8.
Wininger, J. D. and Kort, H I. (2002) Cryopreservation of immature and mature human DOC) tes. Semin. Reprod Mcd, 20, 45-49.
Winslow, K. L., Yang, D., Blohm, P. L., Brown, S. E., Jossim, P. and Nguyen, K. (2001 Ooyte cryopreservation-a three year follow-up of sixteen births. Fertil. SteriL, 76 (Supp 1), S120 (abstract).
Wu, J., Zhang, L. and Wang, X. (2001) In vitro maturation, fertilization., and embryo d pircnt after ultrarapid freezing of immature human oocytes. Reproduction, 121, 389-393.
Yang, D. S., Winslow, K. L. and Blohm, P. L. (1998) Improved survival rate after cryopreservation of human fresh and aged unfertilized oocytes using a specially developed oocyte cryoprese:rvation regime. H'ertil. S.eril., 70 (Suppl. 1), 586.
Yang, D. S., Blobm, P. L., Kramer, L., Nguyen, K., Zhao, Y. L. and Winslow, K. L. (19c)) A successful human oocyte cryopreservation regime: survival, implantation, and pregnancy rates are comparable to that of embryos generated from sibling oocytes. Fertil. Steril., 72 (Suppl. 1), S86 (abstract).
Yang, D., Winslow, K. L., Blohm, P. L., Brown, S. E., Nguyen, K. and Brubaker, C. (2W)₂) Oocyte donation using cryopreserved donor oocytes. Fertil. Steril., 78 (Suppl.), 514 (abstract).
Yoon, T. K., Chung, H. M., Lim, J. M., Han, S. Y., Ko, J. J. and Cha, K. Y. (2000) Pregnancy and delivery of healthy infants developed from vitrified oocytes in a stimulated in vitro fertilization-embryo transfer program. FertiL SteriL, 74, 180-181.
Zenzes M T, Bielecki R, Casper R F, Leibo S P. Effects of chilling to 0 degrees C. on the morphology of meiotic spindles in human metaphase II oocytes. Fertil Steril 2001; 75: 769-77.

1. A sodium-depleted cryopreservation media which does not employ a PBS buffer solution for use with mammalian cells.

2. The media of claim 1 wherein said media contains HEPES buffer.

3. The media of claim 2 wherein HEPES is present in a range of about 10 mM to about 25 mM.

4. The media of claim 2 wherein HEPES is present in an amount of about 21 mM.

5. The media of claim 2 wherein hyaluronic acid is present in a range of about 0.1 mg/ml to about 1.0 mg/ml based on total volume of the cryopreservation media.

6. The media of claim 2 wherein said mammalian cell is an oocyte.

7. The media of claim 6 wherein said oocyte is a human oocyte.

8. The media of claim 2 which optionally comprises a cryoprotectant selected from the group consisting of 1,2 propanediol, dimethyl sulfoxide, glycerol, ethylene glycol, and combinations thereof.

9. The media of claim 3 which optionally comprises a cryoprotectant selected from the group consisting of 1,2 propanediol, dimethyl sulfoxide, glycerol, ethylene glycol, and combinations thereof.

10. The media of claim 9 wherein said cryoprotectant is 1,2 propandiol.

11. The media of claim 10 wherein said cryoprotectant is present in a range of from about 100 to about 2500 mM 1,2 propanediol.

12. The media of claim 1 wherein said sodium is present in an amount less than 7 mM.

13. The media of claim 2 wherein said sodium is present in an amount of from about 1 mM to about 2 mM.

14. The media of claim 1 which optionally comprises hyaluronic acid.

15. The media of claim 14 wherein said hyaluronic acid is present in an amount of from about 0.1 mg/ml to about 1.0 mg/ml based on total volume of the cryopreservation media.

16. The media of claim 1 wherein said media increases the viability of the cryopreserved oocyte.

17. The media of claim 2 wherein said media increases the viability of the cryopreserved oocyte.

18. The media of claim 9 wherein said media increases the viability of the cryopreserved oocyte.

19. The media of claim 10 wherein said media increases the viability of the cryopreserved oocyte.

20. A sodium-depleted cryopreservation media consisting essentially of sodium in a concentration of about 1 mM to about 2 mM, and HEPES in a concentration of about 21 mM.

21. A method of increasing the viability of cryopreserved oocytes comprising freezing said oocyte in the cryopreservation medium of claim 1, wherein the viability of the oocyte upon thawing is increased compared to oocytes which are not cryopreserved in said media.

22. A method of increasing the viability of cryopreserved oocytes comprising freezing said oocyte in the cryopreservation medium of claim 2, wherein the viability of the oocyte upon thawing is increased compared to oocytes which are not cryopreserved in said media.

23. A method of increasing the viability of cryopreserved oocytes comprising freezing said oocyte in the cryopreservation medium of claim 3, wherein the viability of the oocyte upon thawing is increased compared to oocytes which are not cryopreserved in said media.

24. A method of increasing the viability of cryopreserved oocytes comprising freezing said oocyte in the cryopreservation medium of claim 4, wherein the viability of the oocyte upon thawing is increased compared to oocytes which are not cryopreserved in said media.

25. A viable oocyte which has been cryopreserved using the media of claim 1.

26. A viable oocyte which has been cryopreserved using the media of claim 2.

27. A viable oocyte which has been cryopreserved using the media of claim 3.

28. A viable oocyte which has been cryopreserved using the media of claim 4.

29. The media of claim 1 wherein said media contains MOPS buffer.

30. The media of claim 29 wherein MOPS is present in a range of about 10 mM to about 25 mM.

31. The media of claim 29 wherein MOPS is present in an amount of about 21 mM.

32. The media of claim 29 wherein hyaluronic acids is present in a range of about 0.1 mg/ml to about 1.0 mg/ml based on total volume of the cryopreservation media.

33. The media of claim 29 wherein said mammalian cell is an oocyte.

34. The media of claim 33 wherein said oocyte is a human oocyte.

35. The media of claim 29 which optionally comprises a cryoprotectant selected from the group consisting of 1,2 propanediol, dimethyl sulfoxide, glycerol, ethylene glycol, and combinations thereof.

36. The media of claim 30 which optionally comprises a cryoprotectant selected from the group consisting of 1,2 propanediol, dimethyl sulfoxide, glycerol, ethylene glycol, and combinations thereof.

37. The media of claim 36 wherein said cryoprotectant is 1,2 propandiol.

38. The media of claim 37 wherein said cryoprotectant is present in a range of from about 100 to about 2500 mM 1,2 propanediol.

39. The media of claim 29 wherein said sodium is present in an amount less than 7 mM.

40. The media of claim 29 wherein said sodium is present in an amount of from about 1 mM to about 2 mM.

41. The media of claim 29 which optionally comprises hyaluronic acid.

42. The media of claim 41 wherein said hyaluronic acid is present in an amount of from about 0.1 mg/ml to about 1.0 mg/ml based on total volume of the cryopreservation media.

43. The media of claim 29 wherein said media increases the viability of the cryopreserved oocyte.

44. The media of claim 36 wherein said media increases the viability of the cryopreserved oocyte.

45. The media of claim 37 wherein said media increases the viability of the cryopreserved oocyte.

46. A sodium-depleted cryopreservation media consisting essentially of sodium in a concentration of about 1 mM to about 2 mM, and MOPS in a concentration of about 21 mM.

47. A method of increasing the viability of cryopreserved oocytes comprising freezing said oocyte in the cryopreservation medium of claim 29, wherein the viability of the oocyte upon thawing is increased compared to oocytes which are not cryopreserved in said media.

48. A method of increasing the viability of cryopreserved oocytes comprising freezing said oocyte in the cryopreservation medium of claim 30, wherein the viability of the oocyte upon thawing is increased compared to oocytes which are not cryopreserved in said media.

49. A method of increasing the viability of cryopreserved oocytes comprising freezing said oocyte in the cryopreservation medium of claim 31, wherein the viability of the oocyte upon thawing is increased compared to oocytes which are not cryopreserved in said media.

50. A method of increasing the viability of cryopreserved oocytes comprising freezing said oocyte in the cryopreservation medium of claim 32, wherein the viability of the oocyte upon thawing is increased compared to oocytes which are not cryopreserved in said media.

51. A viable oocyte which has been cryopreserved using the media of claim 29.

52. A viable oocyte which has been cryopreserved using the media of claim 30.

53. A viable oocyte which has been cryopreserved using the media of claim 31.

54. A viable oocyte which has been cryopreserved using the media of claim 32.
